(19) United States
(12) Patent Application Publication
(10) Pub. No.: US 2022/0068450 A1
(43) Pub. Date: Mar. 3, 2022

(54) INTEGRATING MANAGEMENT OF MOVEMENT SESSIONS WITH ELECTRONIC RECORD SYSTEM REPORTING REQUIREMENTS

(71) Applicant: Xperience Robotics, Inc., Virginia Beach, VA (US)

(72) Inventor: Ankit Shah, Virginia Beach, VA (US)

(21) Appl. No.: 17/458,103

(22) Filed: Aug. 26, 2021

Related U.S. Application Data
(60) Provisional application No. 63/070,773, filed on Aug. 26, 2020.

(51) Int. Cl. G16H 15/00; G16H 10/60; G16H 40/20; G16H 40/67; G16H 20/30; A63B 24/00

(52) U.S. Cl. CPC G16H 15/00; G16H 10/60; G16H 40/20; A63B 2220/836; G16H 20/30; A63B 24/0062; A63B 24/0075; G16H 40/67

(57) ABSTRACT

A method of integrating management of subject movement sessions with reporting requirements for an electronic health records system includes receiving subject information with a server of a device system, and generating movement plan options for the subject based on the subject information. Parameter measurements are determined from raw data obtained by sensors of wearable devices of the device system worn by the subject during performance of movements of a movement plan selected from the movement plan options. Metrics values are identified or converted from the parameter measurements as part of the method. The metrics values correspond to reporting metrics required by the record system. The server or a computing device generates a report according to a format of the records system including the metrics values, the report including at least the parameter measurements identified as the metrics values for the subject.

### CROSS REFERENCE TO RELATED APPLICATIONS

This application claims the benefit of priority under 35 U.S.C. § 119(c) to U.S. Provisional Patent Application No. 63/070,773 entitled “INTEGRATING MANAGEMENT OF MOVEMENT SESSIONS WITH ELECTRONIC RECORD SYSTEM REPORTING REQUIREMENTS,” filed Aug. 26, 2020, which is herein incorporated by reference in its entirety for all purposes.

### BACKGROUND

Today, many industries, e.g., healthcare, banking and finance, manufacturing, and others, have extensive reporting requirements. These requirements may be instituted by insurance companies, government bodies or agencies, recognized associations, and/or license granting or monitoring organizations. Various types of electronic record systems are used across and within these industries.

The healthcare industry, for example, is heavily reliant on electronic record systems, also known as electronic health record (“EHR”) or electronic medical record (“EMR”) systems. Examples of such EHR systems can include KAREO, WEBPT, various electronic medical record software products from vendors such as CERNER HEALTH and EPIC, and the like. These systems are used to facilitate the provisioning of treatments, surgeries, equipment, or the like to insurance policy holders at costs commensurate with the specific terms of respective policies. At the same time, insurance companies rely on these electronic record systems to ensure claims for payment of services are not fraudulent, or outside the scope of policy terms.

These electronic record systems in the healthcare industry can provide some degree of certainty in the payment of medical bills, and ensure some level of legitimacy to claims. However, meeting reporting requirements is tedious, time consuming, and at times, confusing. This places a tremendous labor and time burden on healthcare providers, in all areas of healthcare, that must meet these reporting requirements to obtain payment for the services they provide. All too often, this negatively impacts the time and care with which these individuals may be able to provide their services to subjects, and thus the quality of those services. At the very least, providing quality services is made more difficult due to the reporting requirements that if not done correctly, negatively impacts a healthcare provider's ability to be compensated for the health-related services they provide.

The issue described above, at least within the healthcare industry, is present in full effect for physical and rehabilitative therapies. Physical therapists, physical therapy assistants, interns, or students (together referred to hereafter as “PTs” or “PT”), often provide treatment by guiding, assisting, correcting, and/or aiding in some other way, the motion of subjects performing, or attempting to perform, prescribed movements. The prescribed movements being part of respective movement plans designed to strengthen, improve the range of motion of, or otherwise rehabilitate a target area of the subjects' bodies.

To be compensated for the services they provide subjects, PTs must document the movements performed during movement sessions and measurements of the subjects' performance according to specific parameters (e.g., overall range of motion, number of repetitions, severity of pain during movement, heart rate during movement, level of tension in a muscle receiving targeted treatment, etc.). Thus, for a given movement session, the PT must have some way of keeping track of what movement a subject is doing and how well the movement is being performed, and then reporting this information to a subject's particular insurance provider.

A level of detail and degree of accuracy of session information provided by a PT may be factors an insurance company may use as basis to withhold payment. In addition, whether or not the information is accurate can impact a subject's ability to have additional therapy covered by a insurance provider. For example, if a PT reports that a subject has 80% range of motion with their arm, while in reality the subject can only move their arm over 60% of a normal range of motion, the insurance company may determine additional therapy is no longer needed. Still further, for a movement session to fall under a particular subject's policy, and thus allow for a PT to be compensated, certain insurance companies may require certain movements be done during movement session and reported on.

It is often the case that PTs treat multiple subjects having different movement plans at the same time. For example, it is not uncommon for a PT to be, all at the same time: (1) monitoring the motion of subject's arm about their shoulder; (2) physically guiding the flexion and extension of another subject's leg at their knee; and (3) instructing a third subject to hold a resistance band at one end while flexing and extending their foot around which the other end of the band is wrapped. Unfortunately, this situation is often necessitated by decreasing amounts of compensation that insurance companies pay for PT services. But whatever the reason for the volume of subjects, a reporting requirement for each, and a burden of a PT to meeting those requirements, remains same.

As a result of reporting requirements, PTs often spend in-session time on laptops or other devices focused on documenting subject movements an performance parameter measurements. However, physical therapy as a field, is interpersonal, and success often depends in large part to a quality of a human element incorporated in the services being provided. The reporting requirements that are critical to a PT's ability to get compensated and continue providing their services can negatively impact the human element each PT brings to the service they provide their subjects. Those that try to do both well risk burning out. Further, it is harder for subjects to form connections with these providers and get the most from the providers' services.

As a result, a need exists for systems and methods that permit healthcare providers to focus on subject as they provide services to those subjects, while reporting requirements that must be met are fulfilled accurately without significant attention from the healthcare providers.

### SUMMARY

Examples described herein include systems and methods for integrating the management of movement sessions for subjects with reporting requirements required by an electronic health records system (“records system”) such that healthcare providers such as physical therapist can remain engaged with subjects as services are provided and movements are performed. In one example, a server for a device system can receive subject information and a request for metrics information from the records system. The subject information may correspond with a subject that is associated with the metrics information of the request. Movement plan options (e.g., exercises targeting certain parts of the subject's body for improvement) can be generated by based on the subject information.

As a subject performs movements from a movement plan, raw sensory data (“RSD”) can be obtained by sensors of wearable devices that are part of a device system. The wearable devices may be worn by the subject as the RSD is received, in one example. The RSD may be generated by the wearable devices implementing a training mode during the movement session. Further, a computing device included in, for example, the wearable devices may determine parameter measurements based on the RSD. The parameter measurements may correspond to performance parameters (e.g., number of sets or reps, range of motion, level of pain experienced, time amount for single performance of movement, etc.) for movements included in the movement plan.

In another example, an integration agent can identify, from the parameter measurements, metrics values that correspond to reporting metrics required by the record system (e.g., range of motion, change in range of motion or number of sets or reps from previously performed sets or reps). At least one of one of the server and the computing device can cause a report to be generated according to a format of the records system. In one example, the report can include at least the parameter measurements identified as the metrics values for the subject.

In another example, a report may be submitted through an integration module for the device system based on an authorization received by the integration module. The integration module may be implemented as a graphical user interface (“GUI”) for the device system. In yet another example, the integration module can transmit and receive information to and from a records system and a device system including wearable devices. In one example, the integration module may be launched within a device system interface. In another example, the integration module may be launched within a records system interface.

The examples summarized above can each be incorporated into a non-transitory, computer-readable medium having instructions that, when executed by a processor associated with a computing device, cause the processor to perform the stages described. Additionally, the example methods summarized above can each be implemented in a system including, for example, a memory storage and a computing device having a processor that executes instructions to carry out the stages described.

Both the foregoing general description and the following detailed description are exemplary and explanatory only and are not restrictive of the examples, as claimed.

### BRIEF DESCRIPTION OF THE DRAWINGS

FIG. 1 illustrates front views of an operator and first, second, and third subjects each with wearable devices and engaged in respective movement sessions as part of an exemplary implementation of a system for integrating management of movement sessions with reporting requirements for an electronic heath records system (“records system”).

FIG. 2 is a flowchart of an exemplary method for integrating management of movement sessions with reporting requirements for a record system.

FIG. 3A is a sequence diagram of an example method for launching a device system (“DS”) interface.

FIG. 3B is a sequence diagram of an example method for identifying and converting metrics values from parameter measurements collected during movement sessions.

FIG. 3C is a sequence diagram of an example method for a records integration that includes integrating identified or converted parameter measurements into a record system.

FIG. 4 is a sequence diagram of an example method for integrating management of a movement sessions with reporting requirements of a records system.

FIG. 5A illustrates an exemplary graphical user interface (“GUI”) for monitoring a movement sessions of subjects in real time.

FIG. 5B illustrates an exemplary GUI for comparatively monitoring a movement session of a subject in real time using a computing device.

FIG. 5C illustrates an exemplary GUI for generating and reviewing record-system ready data within a dashboard during or following a movement session.

FIG. 6 is an exemplary illustration of system components for integrating movement session management with reporting requirements for a records system.

### DESCRIPTION OF THE EXAMPLES

Reference will now be made in detail to the present examples, including examples illustrated in the accompanying drawings. Wherever possible, the same reference numbers will be used throughout the drawings to refer to the same or like parts.

Reference numerals and terminology used herein are for the purpose of describing particular aspects only and are not intended to be limiting. For example, as used herein, the singular forms “a,” “an” and “the” are intended to include the plural forms as well, unless the context clearly indicates otherwise. As another example, recitation of ranges of values herein are merely intended to serve as a shorthand method of referring individually to each separate value falling within the range. Unless otherwise indicated herein, and each separate value is incorporated into the specification as if it were individually recited herein.

It is noted that any one or more aspects or features described with respect to one example, may be incorporated in different examples described herein, although not specifically referred to or otherwise described relative thereto. That is, all examples and/or features of any aspect described herein can be combined in any way and/or combination. Thus, all methods described herein can be performed in any suitable order unless otherwise indicated herein or otherwise clearly contradicted by context.

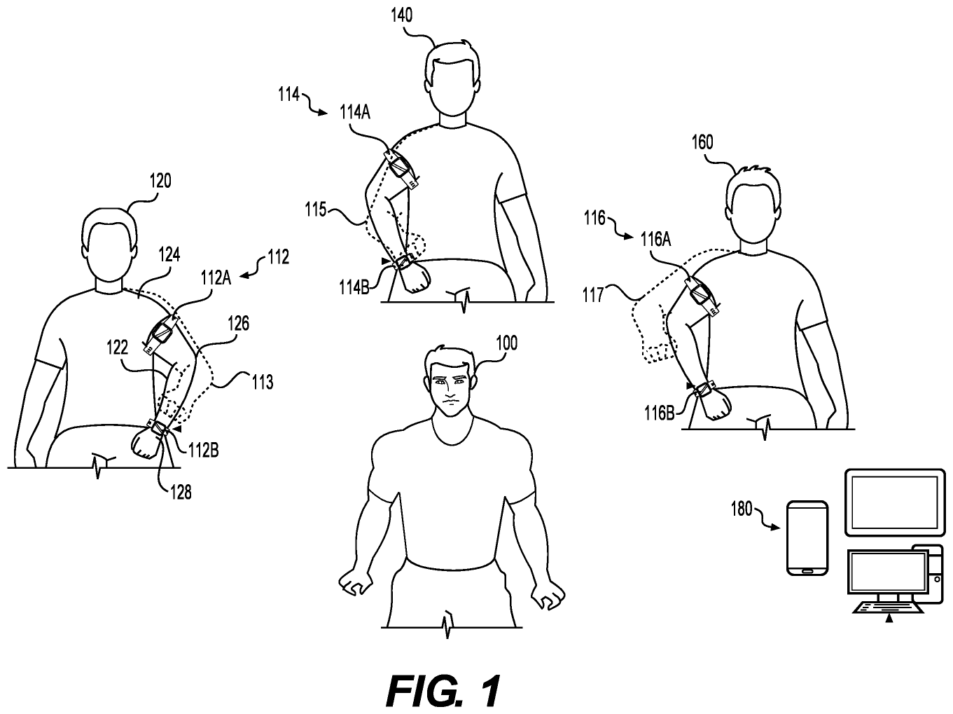

FIG. 1 illustrates front views of an operator 100 and a first subject 120, a second subject 140, and a third subject 160. Each of the first, second, and third subjects 120, 140, 160 are in the process of performing a movement included in each subject's respective movement plan. The correctness, and hence the effectiveness, of each subject's attempt to perform a respective movement is determined with reference to a first, second, and third movement model 113, 115, 117 respectively.

More specifically, the first subject 120 is shown wearing first and second wearable devices 112A, 112B of a first device group 112 of a device system. These devices sense the first subject's movement and compare it to a first movement model 113. Likewise, the second subject 140 is shown wearing third and fourth wearable devices 114A, 114B of a second device group 114 of the device system. These devices sense the second subject's movement and compare it to a second movement model 115. Finally, a third subject 160 is shown wearing fifth and sixth wearable devices 116A, 116B of a third device group 116 of the device system. These devices sense the third subject's movement and compare it to a third movement model 117.

According to an aspect of the present disclosure, “wearable device” includes any form factor designed or intended to be worn by a person (e.g., personal equipment such as helmets, face guards, apparel, or the like; personal devices such as head-mounted electronic devices, wrist mounted devices, body-mounted devices, devices worn around the neck; or any form factor that, while not necessarily designed or intended to be worn by a person, may be adapted to be worn by a person (e.g., smartphones, tablet computers, and/or other digital processing devices).

Each wearable device may constitute a computing device including a processor, a memory storage, and a non-transitory computer-readable medium containing instructions that are executed by the processor. In addition, each of the wearable devices includes a plurality of motion and other types of sensors installed in the device and in communication with the processor. In one example, a wearable device, such as the first and second wearable devices 112A, 112B, may include at least a gyroscope, an accelerometer, and a magnetometer. In another example, a wearable device such as the first and second wearable devices 112A, 112B, may include a hall sensor, optical encoders, and/or any form of optical sensor for example.

Any wearable device described herein may include a component that authenticates biometric-information (“bio-metric component”), a pulse oximeter, a blood oxygen sensor, and/or an iris scanner. This biometric component or sub-component may acquire biometric information from a subject and use the acquired biometric information to authenticate and permit the subject to use the wearable device. The biometric component may acquire biometric information through a surface or portion of the wearable device that is particularly close to or in contact with the subject. The biometric information may be defined as information associated with the subject's living body (e.g., pulse, fingerprint, movement profile of a one portion of the subject's body, body temperature, etc.) the face, and the walking pattern. This configuration may biometrically identify and authenticate the subject without requesting an action by the subject.

Further with regards to hardware, any wearable device described herein can include a screen. In one example, a screen could be provided as a device's interface with, and a vehicle for delivering feedback to, a subject wearing the device. In another example, versions of a wearable device provided with a screen (e.g., an LCD screen, a touch sensitive screen, and the like), as well as versions that do not include a screen, may include LEDs, vibration motors, and/or a means of generating some type of audio feedback.

Any wearable device according to the present disclosure can be capable of transferring data to or receiving data from another wearable device in a device group, as well as performing the methods described herein for delivering real-time model compliance feedback. However, distribution of certain processing functions may be placed with one device over the other for a particular device group due to where that device will be positioned, what designated movements are part of a movement plan, and other factors. The wearable devices for a device group according to the present disclosure can communicate with each other using any wireless protocol (e.g., Bluetooth, Bluetooth Low Energy, Zigbee, or a Wifi chip—this option would allow for direct communication with, for example, a cloud).

As used herein, the term “real-time,” “real time,” and “substantially real-time” are used to refer to processing performed within strict time constraints. For example, as described herein, real-time tracking, collection, processing, and delivery of raw sensory data, formatted data, comparative analysis data, and feedback: (1) can be performed; (2) as an event triggering that performance occurs; (3) without a perceivable delay after the occurrence of the event.

Each of the wearable devices in each of the first, second, and third device groups 112, 114, 116 may communicate directly with other computing devices (e.g., a phone, tablet, laptop, or desktop computer) such as a computing device 180 of FIG. 1, and/or over a network such as the internet, or in wired or wireless fashion, using one or more components. In one example, any of the wearable devices 112A, 112B, 114A, 114B, 116A 116B can communicate with an external device, such as a phone, and the external device can simultaneously transmit the raw sensory, formatted, and/or comparative analysis data received or generated, to a cloud or a backend of a system through a network connection.

As a subject, such as the first subject 120, performs a movement included a movement plan, feedback that can be delivered to the first subject 120 by the first and second wearable devices 112A, 112B. In one example, a type of feedback can be specific to a device based on its proximity to a portion of a subject's body that may deviate more substantially from a movement model, than any other body portion part of a kinematic chain involved by the designated movement. In another example, a type of feedback can be specific to a device based on its proximity to a portion of a subject's body that, based on a kinematic chain involved, has a more prominent role in an overall deviation from a movement model.

FIG. 1 is included to provide a visualization of how a movement models can differ. Each of the first, second, and third movement models 113, 115, 117 are tailored to the first, second, and third subjects 120, 140, 160 respectively based on that subject's abilities. A focus of a movement model, for example the first movement model 113, accounts for a kinematic chain involved in the designated movement with respect to the first subject 120. Thus, the first movement model 113 is not merely representative of an intermediate goal between movement profiles of the first subject 120 and an optimal movement pattern for someone lacking a condition that affects that person's ability to do an arm raise. Rather, it represents the specific movements, positions, and/or points of emphasis or exertion for particular segments in the first subject's kinematic chain (e.g., should 124, elbow 126, shoulder 128) that the first subject 120 may need to focus on to incrementally change how that kinematic chain operates.

For example, complying with the first movement model 113 at a stage of a movement plan for the first subject 120 may require the first subject to focus on maintaining a shoulder level throughout a range of motion for an arm raise. Subsequently, a new version of the first movement model 113 for this designated movement may cause the first subject 120 to bend his elbow less at a first time than a previous version of the movement model 113. However, it may not be until the first subject 100 maintains a shoulder level until the device system generates this subsequent version of the first movement model 113.

In FIG. 1, each of the first, second, and third subjects 120, 130, 140 are in the process of performing an arm raise. For example, the first subject 120 has started moving an arm 122 to perform a designated arm raise. However, the subject's arm 122 deviates from the first movement model 113 at each of the three joints, the shoulder 124, elbow 126, and wrist 128, involved in the movement. As a result, each of the first and second wearable devices 112A, 112B can be delivering specific types of feedback to the first subject. The first wearable device 112A located near the first subject's shoulder 124, can deliver a first feedback in the form of a flashing touch screen. On the other hand, a second feedback delivered by second wearable device 112B positioned near the wrist 128 can include a rapidly repeating pattern of three-pulse vibrations.

The computing device 180 also illustrated in FIG. 1, implements at least one service of an electronic records system. Each of the first, second, and third device groups 112, 114, 116 are configured to have their management of movement sessions by their respective subjects, integrated with reporting requirements stipulated by, through, or otherwise in accordance with the electronic records system.

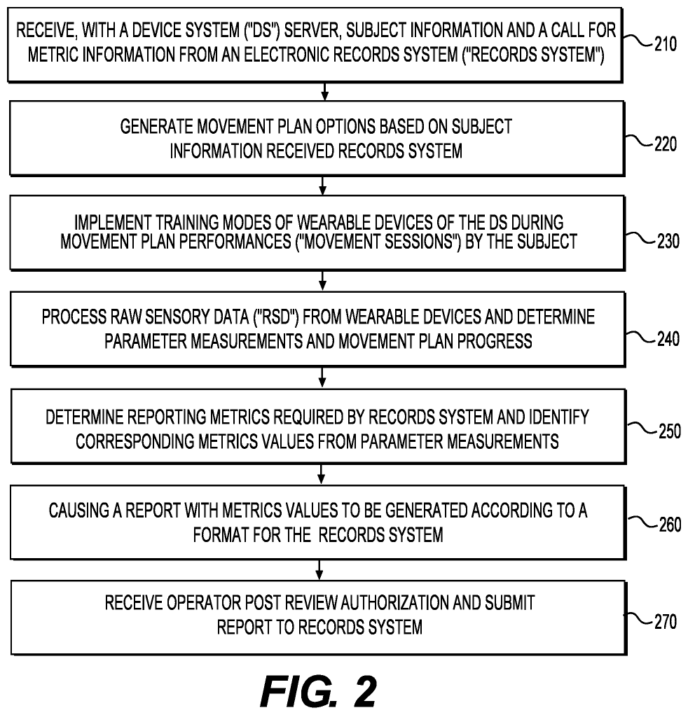

FIG. 2 is a flowchart of an exemplary method for integrating management of movement sessions with reporting requirements for a record system.

At stage 210, a device system server (“DS server”) can receive subject information and a call for metric information from an electronic records system (“records system”).

The subject information can be retrieved by record system upon selection of an option within a record system interface to access the device system. In on example, the record system interface may include an option to launch a device system interface on a computing device where the record system interface is being implemented or accessed. This can be implemented through an application programing interface (“API”) call from a backend of the record system to a device system backend.

In one example, the API call can be initiated by the selection of device system option within the record system interface. The API call can be communicated to a device system backend through the record system backend which supports the record system interface being accessed on a user device, for example. The record system backend may be in communication with the device system backend over a network. In another example, the record system backend may be configured to open a socket connection with the device system backend. In another example, a secure socket connection or communication channel can persist between the record and device system backends. In another example, the API call could be implemented locally on a user device, through web applications and/or cloud-computing based communication protocols between the two systems.

In stage 220, the DS server can transmit the subject information to a user device configured to communicate and configure wearable devices of the device system. The subject information can be used to understand a subject's condition and generate movement plan options based on subject information received.

At stage 230, the device system can implement a training mode with wearable devices of the device system during a movement plan performance (“movement sessions”) by the subject. A training mode can be implemented according to a training mode as disclosed in U.S. patent application Ser. Nos. 16/859,005 and 16/859,046, which are expressly incorporated by reference in their respective entireties for all purposes.

In stage 240, raw sensory data (“RSD”) from wearable devices can be processed and used to determine performance parameter measurements and movement plan progress. In one example, the wearable devices include a plurality of movement sensors.

At stage 250, the device system access a library of movements to determine reporting metrics required by the records system for a subject's condition. In one example, the library is stored as a database in, or is on a server that is accessible to, the DS server. In another example, the device system may determine the reporting metrics at the same time the subject information is received. The device system uses the reporting metrics information to determines which of the performance parameters, for which measurements are being or have been determined from the RSD, correspond to the reporting metrics.

In one example, a performance parameter may be comprised of sub-parameters, only some of which correspond to reporting metrics. Accordingly, the device system will identify values of the corresponding sub-parameters for the reporting metrics. For example, during an arm raise a clinician, with or her own hand or arm, may oppose the upward motion of the patient's arm as part of a stability exercise. The wearable devices may be calibrated to determine or estimate the amount of force is being applied to the patient's arm by the clinician's impedance. Further, the device system may be calibrated to record an amount of time the force is applied, a sub-range with a total range of the patient's arm motion the forces was applied, as well as a speed of the patient's motion over that sub-range. The performance parameter the device system may register may be, for example, a ratio between (1) an angular velocity of the patient's arm over the sub-range, and (2) foot-lbs of force applied by the clinician. The reporting metric however, may only be concerned with an amount of time, or range values for the sub-range, that the arm's motion was opposed. The device system can therefore breakdown the components of the velocity to force ratio it has registered for the subject, into values for the required reporting metrics.

In stage 260, the device system can prepare a report with metrics values identified or converted from the performance parameter measurements according to a format for the records system. In one example, the format of the report may be determined from the movement library. In another example, the DS server can receive the format when the subject information is received. In yet another example, the format of the report can represented by a collection of programing language variables provided in a file of a file format, for example a JSON or XML, file format. The device system can be configure to generate a file in a file format required by the records system.

In still another example, the format required by the records system may be a file format for which the parameter measurement/reporting metric information can be presented according to that file format in a user interface. The user interface being provided by an integration service with access to a file in the required format. The integration service could be a part of the device system and/or the records system, but is at the very least compatible with both. In various examples, the integration system may be implemented on a computing device in communication with, or otherwise implementing some component of, the device system and/or the record system.

Continuing with the example immediately above, prior to submission in stage 270, the performance parameter measurements corresponding to required reporting metrics can be displayed in the required format within a module or component of a device system interface and/or a records system interface. The device system interface, which is normally used for calibrating the wearable devices, building subject-specific movement plans, checking the subject's movement plan progress, and communicating with the subject, can include a module or other service component that generates a report in the format that may be accepted by the record system for processing. Alternatively, or in addition to, the device system may support a device system specific module within the records system that can be activated with an API call from the device system to the record system. Following, or as part of, the API call, the device system can transmit the relevant performance parameter measurements corresponding to the required reporting metrics in the required format over a network, web sockets, a secure data channels, or through a local data transfer on a computing device between services and a subsequent accessing of a backend or other server associated with the record system.

In another example, the format for the required reporting metrics can include or specify that metric values must be provided in secure documents (e.g., .pdfs). The document may have to a finalized un-editable or even signed document. The document may have to include certain data entry fields presented in a certain order, certain measurement units, and other requirements as to constitute or be an acceptable representation of a standard form used by the records system.

At stage 270, authorization to submit the formatted report can be received, and the report submitted for processing by a management service of the records system. The authorization may be received after a review of the report by a clinician, for example, form the device system and/or record system interfaces. In one example, an interface displaying the report may be configured to enable an operator or clinician to change or edit the report being reviewed.

In another example, where the report is in the form of a finalized document (e.g., a pdf), and there is a discrepancy between the information in the document and aspects of movement session(s) it contains required reporting metric for, an option to delete the report can be provided. In this example, where the report is presented and declined for authorization in a record system interface, an API call can issued to the device system backend, or the device system interface can otherwise be called or relaunched, and the information from the report can be presented in an editable format within the device system interface. In one example, and integration service implemented by the device system can populate a record system integration console with the device interface with the report information for editing. Once changes have been made, a revised report can be generated within the device or record system interfaces prior to submission being authorized.

In some examples, authorization and subsequent submission can include the format report being stored with a patient record for the subject for which the report was generated. Accordingly, the formatted report becomes part of the subject's medical record. Furthermore, the formatted report may be submitted to various insurance-related entities for the purposes of determining payment owed to a clinician that oversaw the movement session that is the subject of the formatted report.

It will be noted that the device system is configured to received information from the record system and determine which subject information is relevant to the device system for accessing records and movement plans associated with that subject. In addition, the device system is configured to recognize, isolate, format, and transmit movement session information that can be accepted, processed, and submitted for insurance-reporting purposes by the record system.

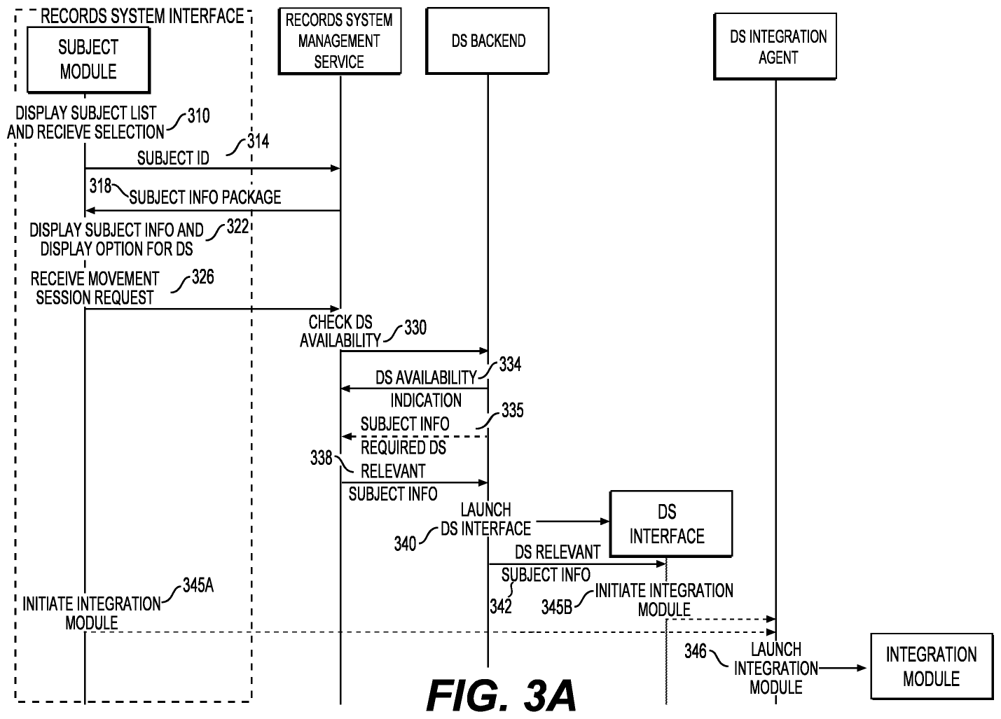

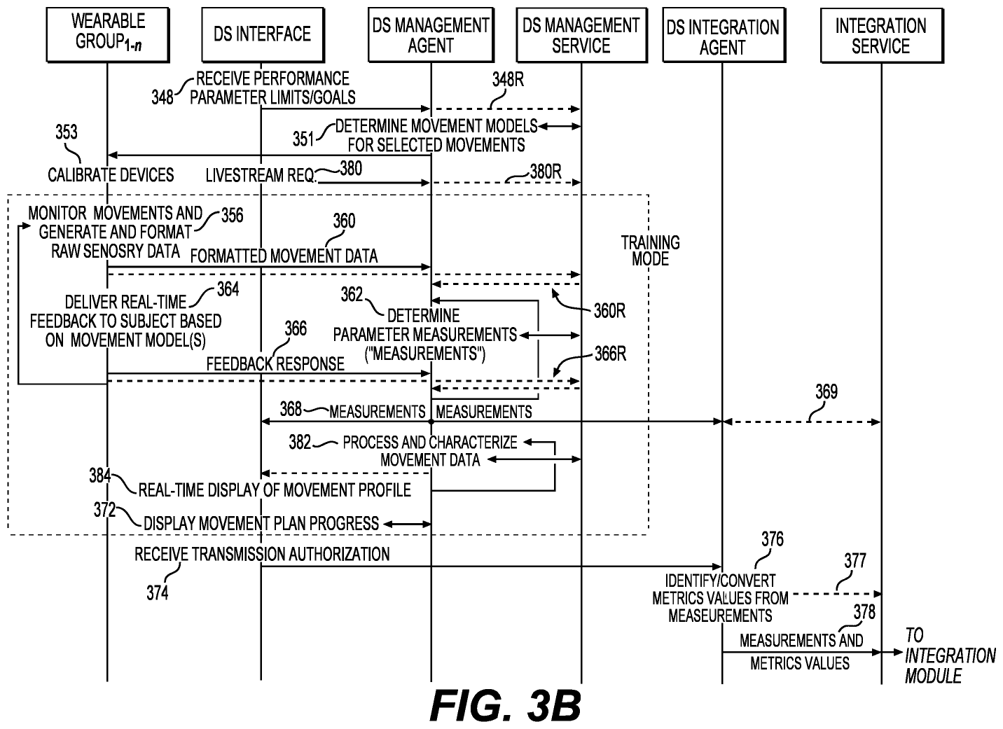

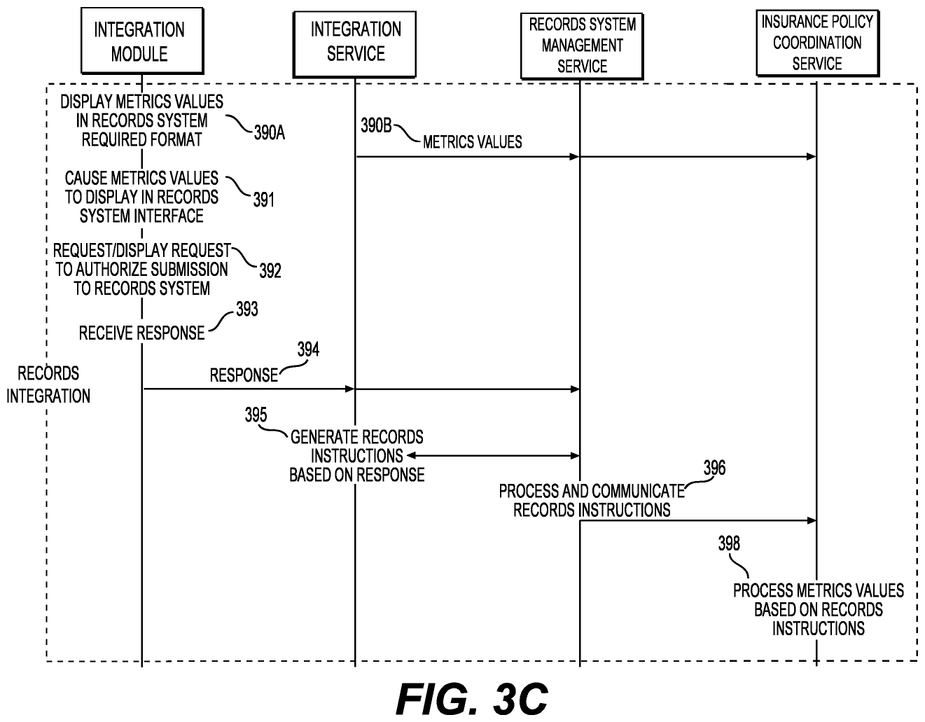

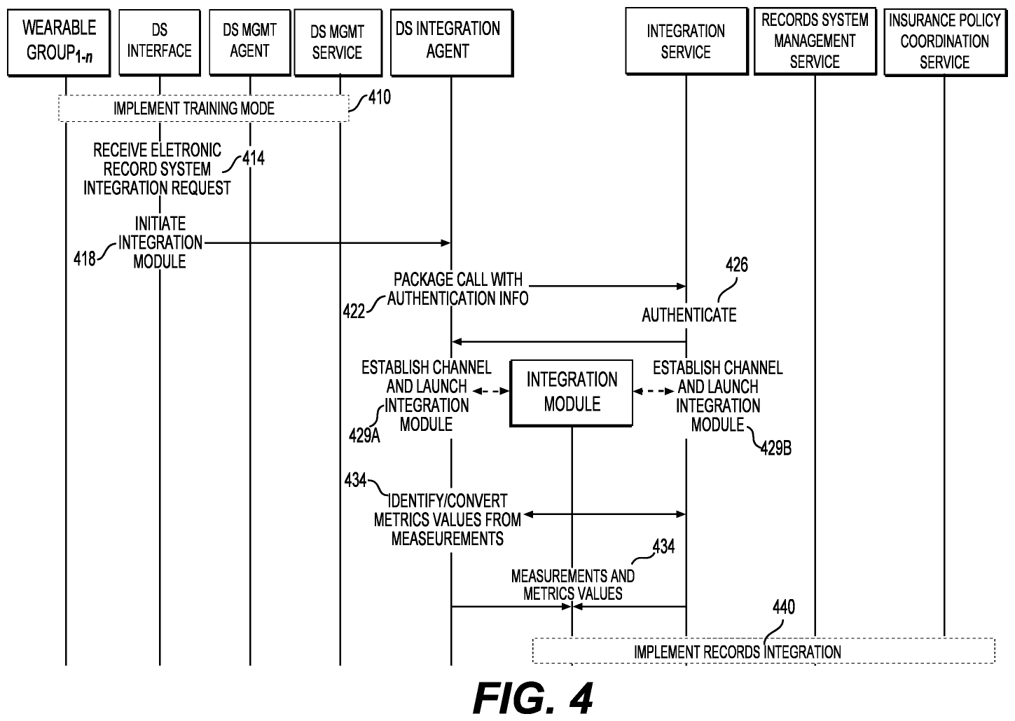

FIG. 3A is a sequence diagram of an example method for launching DS interface. It will be noted that FIG. 3A, as well as FIGS. 3B, 3C, and FIG. 4 provide example methods that can be implemented by a single operator for multiple subjects at the same time or in close temporal proximity with respect to individual subjects. Accordingly, any stage involving an interaction with, or information being generated or communicated with respect to, “a subject” or “the subject,” includes an application of that stage to multiple subjects. For example, with respect of FIG. 3A, stages 314 to 342 refer to a subject and aspects related to a (e.g., subject ID, info package, a movement session request, relevant info), but each of these stages could implemented, iteratively for example, for each subject in a group of subjects. Sor for example, in stage 318, an info package corresponding to a subject ID, which corresponds to one subject, is transmitted to a subject module from a records system management service. If stage 314 include the subject ID of five subjects, stage 318 may be executed five times as the records system management service pull the subject info packages corresponding to the subjects ID of stage 314.

In another example, the execution of any one particular stage could include collection of data or other required inputs for each subject in a group of subjects specified in stage 314 before a central operation executed or implemented for that stage. Thus, staying with stage 318, the system could wait until info packages for all of the subjects requested in stage 314 were found.

At stage 310, a clinician, care provider, or clinical operator (e.g., a medical intern, volunteer, health care administrator) can access a subject module of a record system interface. In one example, a list of subjects can be displayed in a subject module for a records system. The records system management service (“RSMS”) can include a service, application, or other administrator tool executed on a server for the records system. Further, the RSMS can control or otherwise coordinate communication and transfer of information between the records system and other systems, such as the device system or computing and reporting systems for various private and government-associated insurance programs.

A module may be embodied by a separate page, window, or graphical component of an interface described in herein. In addition, a module can comprise computer code, routines, programs, objects, components, data structures, etc. that perform particular tasks or implement particular abstract data types. Further, module may be embodied by a separate page, window, or graphical component of an interface described in herein. In addition, a module can comprise computer code, routines, programs, objects, components, data structures, etc. that perform particular tasks or implement particular abstract data types. Thus, a module can include a set of instructions for performing one or more functions described herein, and may or may not be implemented as separate software programs, procedures, GUI components, or separate individually launchable interfaces modules. Also, a module can comprise a process or sub-process that runs in a background of, for example, a device system interface. This type of module can be responsible for providing specific elements of a GUI that represent information from various systems different from a system immediately represented by the GUI. Thus, a module can be constituted by a routine carrying out a particular task or realize particular abstract, program, object, assembly, data structure, or graphical interface component, or the like.

In stage 314, the records system interface can transmit a subject ID to a record system management service. The subject ID will correspond to a selection received by the subject module of the records system interface. The subject ID corresponds to a subject (also referred to as a person, patient, or healthcare recipient) who has a medical record of some sort in stored in a database, for example, of the records system to the extent that subject was listed in the list of subjects displayed in the subject module.

At stage 318, the record system management service can look up the subject ID in its database or other type of electronic repository of subject records (e.g., a cloud-based solution), and retrieve a package of information including records associated with the subject. In one example, the subject ID can be a subject's name, and the subject info package can include some type of identifier specific to that subject and used by the records system management service to add to a total package of records associated with the subject. In another example, other information can be return in stage 318 such as a condition (e.g., terminal illness, physical injury or other impairment) that may be selected base on information provided in stage 314. For example, the subject ID can be coupled with information that describes who or why the subject info is being requested (e.g., for movement sessions, by a physical therapist, etc.)

In stage 322, the subject information package can be displayed in the subject module and a selectable option to launch a device system can be displayed in the subject module. In one example, the option can include some indication that a movement session can be launched from the subject module. Following display of the option, a request to start a movement can be received at stage 326, and the request can be passed to the records system management service.

In stage 330, the records system management service can check the availability of a device system. In one example, the records management service can send a status request to a backend of the device system. In another example, the records system management service and the DS backend can correspond back and forth in stages 330 and 334 to ensure that the device system has group of wearable devices available for a movement session. In one example, the DS backend can process a condition of the subject, and determine if a device group with an adequate number of wearable devices is available for a movement session for treating that conditions is available. In yet another example, the DS backend can check to see if there is adequate connectivity between the DS backend and a given group of wearable devices for the purposes of a movement session.

Optionally, at stage 335, the DS backend can respond to the availability check in stage 330 by requesting specific subject information that has not already been provided and is required for a movement session. In all cases, whether or not the DS backend engages in stage 335, the records management service will transmit relevant subject information to the DS backend at stage 338. In one example, this information can include a name, physical characteristics (e.g., height, weight, etc.), a device system ID assigned to the subject that the records system has stored, and a condition/physical capabilities or limitations. This information can be used by the device system, for example, for an assessment of the subject and/or the subject's first movement session managed by the device system.

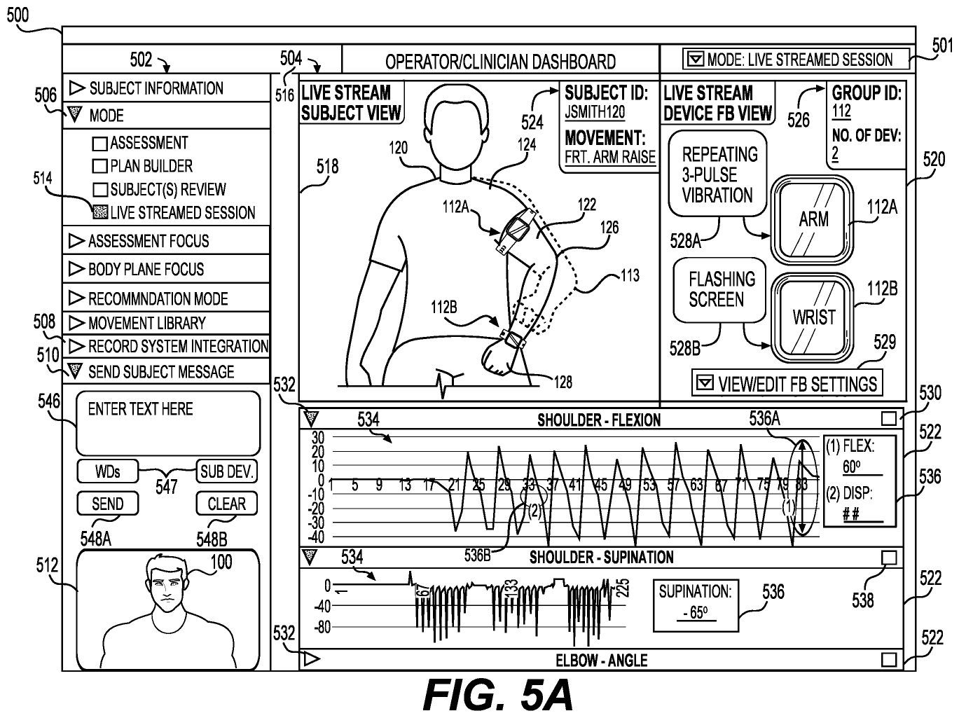

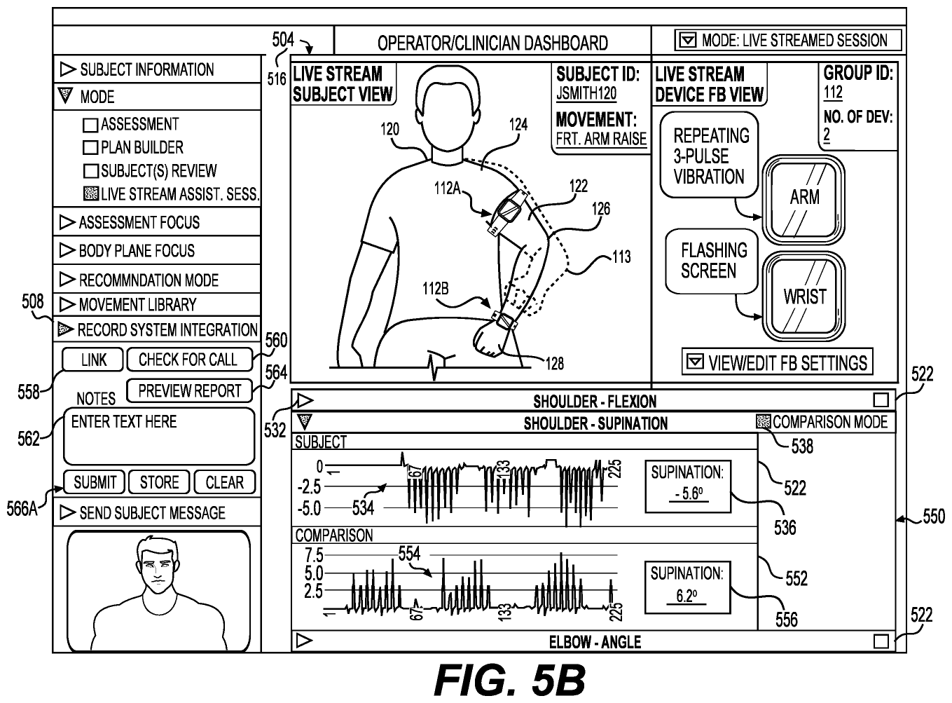

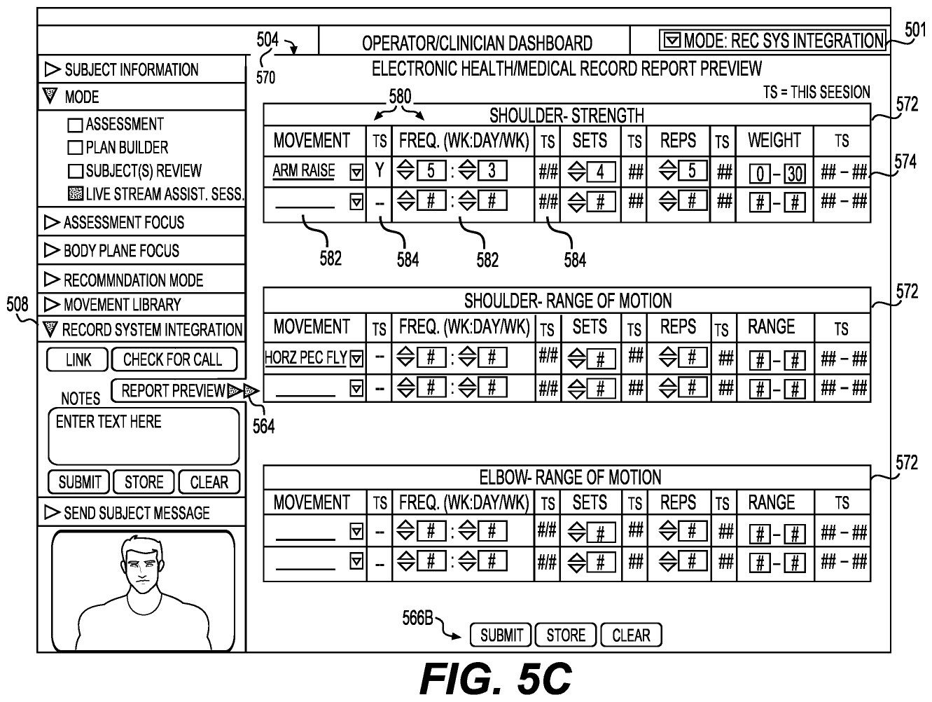

At stage 340, the DS backend can launch a DS interface as result of receiving the device system relevant information in stage 342. The DS interface can include a dashboard, as discussed in more detail with reference to FIGS. 5A-C. Different modes of the system, all of which may require some portion of information provided in stage 338, can be accessed an initiated from the DS interface including an assessment mode or a training mode that executes during a movement session.

In one example, stages 330 to 340 can embody or be aided by a SMART launch in which the records system interface provides an interface component, such as the subject module, from which another application is launched.

In one example, a third party application can be launched from within the records system that supports and implements the records system interface, through the subject module or an option included in the subject module. In this example, the records system interface can be provided by, or embodied as an application (e.g., a desktop application, dedicated mobile application) that can be “SMART launched.” A backend for the records system can validate a SMART configuration of launched application, and allow an application program interface (“API”) call (or calls) to be made from the records system interface to the DS backend. In one example, the DS backend can include a server and/or partially provided through a cloud service that receives the API call from the records system backend. In turn the DS backend can communicate and launch a device system application (e.g., a desktop application, dedicated mobile application) that provides or otherwise embodies the DS interface. Through this launching of the DS interface within the records system interface, a communication channel may be established that allows for the transfer of information between systems as described in more detail with respect to FIGS. 3B and 3C.

In practice, stages 330 to 340 could include an operator opening a desktop application that includes the record system interface with a subject module. The subject module can include an option to launch the DS interface with information regarding a subject listed in the subject module pre-populating certain fields of the DS interface. Once selected the series of communications and information transfers between backends may be executed. The result of these sub-process being the launching the DS interface from within records system interface in stage 340 with subject information corresponding to the subject selected in stage 314. The DS interface can be launched with fields populated with information about the subject, as well as movements determined by the DS backend to potentially be appropriate for treating the subject's condition. Further, the suggested movements can correspond to movements that the records system has been configured to recognize as movements that are acceptable to insurance companies (e.g., covered as in network, out of network, or are approved for reimbursement).

The various portions of subject information that are relevant to a process to be executed by the device system can be transferred to the DS interface in stage 342. This subject information, or portions thereof, can be displayed in the DS interface.

In stage 345A or stage 345B, an integration module can be initiated by the subject module or the DS interface, respectively. The integration module can include a graphical user interface component or programing object that receives, processes, and in some examples, displays, subject information from the record system within the DS interface. In addition, the integration module may provide an interface component or programing object that handles the parameter measurements that have been identified by a DS integration agent as corresponding to metrics values. In another example, the integration module may include a processor or other hardware, and/or a programing object that is configured to process and display information from the device system, and provide a user with an option to have the information be submitted or further processed by the records system. Thus, in one example, the integration module can include a graphical component such as page or tab that is accessible from either the records system interface (e.g., via the subject module) or the DS interface, and allow viewing and/or editing of information that the device system has recognized as corresponding to metrics values.

The integration module according to the present disclosure can be part of, embedded within, or otherwise and exclusive component to the records system interface or the DS interface. However, one of ordinary skill in the art will recognize that the integration module is maintained as an instrument of the device system. Depending on privileges and permissions granted to the device system within the records system, the integration module may be embodied as a separate tab or window that is selectable or launched from the subject module or other element of the records system interface. In another example, the integration module can be provided or otherwise accessed as a tab, window, or sub-feature of the DS interface, as shown in FIGS. 5B and 5C. Whether or not the DS interface or the subject module initiate the initiate the integration module will be determined by the configuration of the device system relative to the records system.

In one example, initiating the integration module in stage 345A or 345B includes the subject module or the DS interface issuing a call to have the integration module that is received by a DS integration agent. In one example, the DS integration agent can include a device-level component, such as an API, device system-specific application, or virtualized device, and an application-level component, such as an API, SDK, or application wrapper. The device-level component can include system level privileges. The application-level component can include privileges in certain applications, such as a web- or dedicated-records system application, which can be developed for operation with one or both of device and records system backends. Reference to the DS integration agent is understood to include either or both of the device-level and application-level components unless otherwise specified.

In on example, the DS integration agent can be implemented on a user device to access selected applications or components of applications. For example, an integration module may be run as part as, or independently from, one or both of a device system or records system interface. Accordingly, depending use-policies enforced by either the device or records system on the user device, the DS integration agent can generate launch instructions for launching the selected applications or components of applications, such as an integration module. The integration module can be implemented on the user device according to the application launch instructions for the DS integration agent. In one example, implementing the selected application can include the DS integration agent launching the integration module from a web or dedicated application. Further, the DS integration agent may facilitate a delivery on a user device of an interface for integration module within the DS interface, or within the records system interface, in a browser, or independently.

In stage 346, the DS integration agent can launch the integration module, for example, on a computing device being operating directly or remotely by an operator that submitted the subject ID in stage 314.

FIG. 3B is a sequence diagram of an example method for identifying and converting metrics values from parameter measurements collected during movement sessions.

At stage 348, the DS interface can receive performance parameter limits or goas for one or more subjects that a clinician is dealing with at that moment. These limits can be transmitted to a DS management agent being implemented on a computing device executing the DS interface. The DS management agent, similar to the DS integration agent, can include a device-level component, such as an API, device system-specific application, or virtualized device, and an application-level component, such as an API, SDK, or application wrapper. The device-level component can include system level privileges. The application-level component can include privileges in certain applications which can be developed for operation with device system backend through a DS system management service that communicates with the DS management agent.

At stages 351 and 353, the DS management agent can initiate a process to prepare one or more wearable device groups ready for a movement session by a subject about use the wearable devices of that device group. Stage 351 can include determining a movement for a selected movement and stage 353 can include calibrating wearable devices of a group or groups of wearable devices to be worn by a subject or subjects specified in stage 314. Model selection and device calibration are not explain in detail but are understood to encompass at least those methods described in U.S. patent application Ser. Nos. 16/859,005 and 16/859,046 related to assessing a subject and determining a movement model specific to the subject.

A training mode with now be described with reference to stages 360 to 372. The training mode of the present disclosure includes all of the features described herein, and encompasses any and all subject matter relating to a training mode described in U.S. patent application Ser. Nos. 16/859,005 and 16/859,046 but not explicitly stated. It will further be noted that the training mode corresponds to the performance of movement session by a subject (or movement sessions performed by respective subjects). Accordingly, stage 360 assumes that a movement session has begun and a subject(s) is performing a movement that is part of his or her movement plan.

At stage 356 wearable devices in a wearable device group (“device group”) monitor movements of a subject wearing those wearable devices. Also, the wearable devices generate raw sensory data in tracking the subject's movement performance. Stage 356 also includes formatting the raw sensory movement data for a comparative analysis with one or more movement models. At stage 360, the formatted movement data is transmitted from the wearable devices to the DS management agent. At stage 364, the DS management agent determines parameter measurements from the formatted data. In one example, parameter measurements are registered with instances of formatted data that correspond to: a start or an end of a repetition, a set, or a movement session; a maximum or minimum of a parameter for testing a subject (e.g., magnitude of resistive force applied to subject, speed of forced movement, range of motion, point of failure) during a repetition or a set; a running average of vital statistics such as heart rate or blood pressure during a repetition, set, or movement session; and other tracked elements.

In one example of stage 362, the DS management agent can communicate, directly or through a network, with a DS management service being implemented on the DS backend to determine which formatted data corresponds to parameter measurements. In another example, the DS management agent does not rely on the DS management service for a determination, but rather a repository for storing the parameter measurements that may later be accessed by the DS integration service or the DS integration agent.

As stage 362 is performed, or some time after, the wearable devices can deliver real-time feedback to the subject based on the wearable devices' analyses of the formatted data with respect to a movement model. In particular, the wearable devices can deliver feedback to the subject that indicates the subject is deviating from a movement profile of a movement model having been specifically configured for that subject. Feedback delivered by the wearable devices can include sound, haptic, and/or visual feedback, as well as explicit voice commands that indicate how the deviation should be corrected. In one example, the DS management agent can complete stage 362 by communicating with the DS management service for any formatted data or feedback response processing to parameter measurements.

At stage 366, the wearable devices record a subject's response to feedback delivered in stage 364 in of data that may correspond to general parameters or feedback specific parameters. For example, the wearable devices can record an elapsed time that feedback is issue, which in turn may correspond to an amount of time taken to correct a deviation from a movement model. Other examples of feedback response specific parameter measurements may include maximum deviation corrected, number of deviations corrected, etc.

As illustrated in FIG. 3B, stage 362 can be performed in the form of a feedback loop that reinitiates after feedback response is received. In one example the loop can continue until the wearable devices transmit an indication in stage 360 or 366 that a movement repetition, set, or session has been completed.

In stage 368, the DS management agent can transmit the parameter measurements from stage 362 to the DS interface for display, and to the DS integration agent. The DS integration agent, in turn, can optionally transmit the measurements to the integration service in stage 369. In one example, the DS integration agent may be configured to drive the integration process described with reference to FIG. 3C and FIG. 4. In another example, the DS integration agent and the integration service may share performance of operations required for integrating parameter measures with reporting metrics.

As a result of receiving the parameter measurements in stage 368, the DS interface can display the parameter measurements in stage 372. This can include show the parameter measurements with respect to the goal and limits received in stage 350. In on example, the display in stage 372 can include a projected versus actual progress comparison for each movement in a movement plan, as well as for an overall plan progress.

Following the conclusion of the training mode with the display at stage 372, an option to authorize or to request transmitting the parameter measurements to the record system (backend) can be selected through the DS interface at 374. This indicates that an operator believes that the correct data form reporting to the records system has been capture for one or more movement sessions for one or more subjects. Also in stage 374 an indication of the authorization is transmitted to the DS integration agent.

At stage 376, the DS integration agent reviews all of the parameters for the parameter measurements to identify those that correspond to reporting metrics for a condition of a subject to which the parameter measurements correspond to. In other examples, the DS integration agent may recognize that some parameters are combinations of certain metrics, while other parameters can be combined to obtain values for other metrics As a result of these recognitions, the DS integration agent can make the appropriate conversions to obtain values required reporting metrics from the parameter measurements. In another example, the DS integration agent may connect with the integration service at optional stage 377 to make these identifications and conversions.

In stage 378, one or both of the DS integration agent and service can transmit both the parameter measurements and the metrics values to the integration module.

In one example of the exemplary method illustrated by the sequence diagram of FIG. 3B, an operator may choose to live stream a movement session, or movement sessions on a computing device using the DS interface. A device system of the present disclosure may be configured to display a movement profile for a subject with the DS interface. The movement profile may be represented by a series of graphs that chart a position, angle, stress, strain, or other measurable physical or physiological metric as a function of time during the assessment, as detected by the wearable devices. In another example, a graphical three dimensional representation of a limb or limb segment of the subject in one or more body planes may be displayed. This capability enables the live stream mode described herein. Stages specific to a live streamed movement session, whether conducted from the same location or a remote location from a subject, are designated with numerals 380 to 384.

At stage 380, prior to a training mode commencing, the DS interface can receive a selection for a live streamed movement session. In on example, an operator can desire to live stream the movements of one or more subjects during their respective movement session. In one example, the device system of the present disclosure can enable an operator to view graphical comparisons between respective movement models and actual movement profiles of one or more subjects as the subjects perform those movements. In another example, an operator and view comparison between the movement profiles and optimum movement profiles instead of, or in addition to, the movement models. This can be done in an in clinic setting, or, as described in more detail with reference to stages 348R, 380R, 360R, and 366R, in a remote or virtualized setting.

In the in-clinic setting, stage 380 is followed by stages 356 to 368 as previously described. After the measurements are transmitted to the DS interface and integration agent in stage 368, the DS management agent can process and characterize the formatted movement data in stage 382. In one example, processing and characterizing of the movement data can mirror the processes performed during an assessment of a subject's motion.

In particular, the movement data can be processed and characterized in graphical form by a modeling service. This characterization is based on a calibration of wearable devices in a wearable device group that was previously generated based on a spatial/frame of reference indicative of how a subject moved. As generated, the wearable devices use the calibration information to establish how to measure the subject's motion with the devices' internal sensors, when to generate feedback, and what feedback to generate. Characterized another way, the wearable devices tune respective internal sensors (e.g., set a sensitivity, sampling rate, storage time, error, feedback triggers etc.) based on the calibration provided by the modeling service. These sensor settings, how they are calibrated, consider a condition of the subject that will be using the wearable devices.

Thus, the wearable devices detect movements and generate sensory data with respective internal sensors with the subject's condition as an influence. Along these lines, the modeling service may utilize predictive charts to use the calibration for a wearable devices' sensors during movement sessions to continuously compare that movement to a movement model previously generated for the subject. The movement model could reflect the fact that a subject has an arthritic elbow or Parkinson's as these factors are used for predictions of the subject's progress with respect to a designated movement, plan, or their condition.

As the wearable devices transmit the movement data a modeling service being implemented under the direction of the DS management service, which is in communication with the DS management agent that operates on a computing device being used by the operator. The modeling service can transmit the processed movement data to the DS interface through a combination of the DS management service and agent in stage 382. Stage 382 is looped as shown to continuously process the looped generation of the measurements as one or more subjects perform movements from their respective movement plans.

In stage 384, the DS interface can real-time display and update the subject's movement profile temporally (1) as the subject continues to perform a movement in the subject's movement plan, and (2) before the wearable devices detect a movement stoppage by the subject at stage. The DS interface can receive the processed data generated as part of stage 382 and display one or more subjects' movement profiles real-time in stage 384, after the start and before the end of one loop iteration of stage 382.

Thus, as subjects perform their respective movement plan movements, and before a movement stoppage is detected, a clinician or an operator can view a graphical representation that pertains to individual body segments involved in a plan movement, and is displayed and updated real-time with respect to actual movement performance. More specifically, real-time generated data that measures select characteristics (e.g., range of motion, amplitude of movement, deflection, flexion, supination, angular displacement, stride length, linear displacement, etc.) of a subject's movement of select body segments, can be received and real-time charted into a format the allows a clinician or operator to analyze the movement of those segments on an empirical basis. This contrasts with only being able to perceive the mechanics of a subject's movement visually, the presentation of which could be obscured by the subject's clothing, or skewed by the subject's positional orientation relative to the clinician or operator viewing the subject.

As a result of the real-time continuously self-updating display of graphical representations of subjects' movement profiles presented in stage 384, the clinician can view, analyze, and request a subject alter some aspect of how they perform a plan movement as performance occurs. In turn, the clinician or operator can gain valuable information as to what potential changes in that subject's movement are needed, and what may need to be added to the subject's movement plan for the subject to ultimately be able advantageously change their movement as intended when the movement plan was generated.

In some examples, the livestream option may be used primarily for movement sessions that are being guided by the operator remotely or “virtually.” For example, a subject at his or her home may position themselves in front of a personal computing device, smartphone, tablet, or Wi-Fi enabled camera and perform movements that are part of a movement session that the operator and the subject cannot be in the location for. Aspects of the live streamed movement session that relate to reporting requirements remain the same. More specifically, a training mode for a remotely guided movement session can be initiated from a records system interface launching the DS interface on an operator's computing device in location remote from a subject, in the same manner as it would if the operator/computing device were in the same location (e.g., an operator's clinic, a subject's home).

The stages of a live streamed movement session performed in a remote setting situation are designated in FIG. 3B with an “R” follow their numeral designation. These are stages have a non-remote setting counterpart and accomplish the same operation or provide the same output at that counterpart. For example, in stage 348R, the DS management service, operating on or by the DS backend receives the performance parameter limits and/or goals that are transmitted to the DS management agent for an in-clinic setting. This is in recognition that the DS management service will either be performing more of the operations to analyze movement data from the wearable devices. Alternatively, the DA management service will, at the very least, provide a pathway between the wearable devices that are in the same location as a computing device operated by a clinician that is guiding one or more movement sessions remotely from were subjects are performing movements as part of a movement session.

Thus, in stage 380R, the live stream request is transmitted to the DS management service. Likewise in stages 360R and 366R, the formatted movement data and feedback responses are transmitted from the wearable devices to the DS management service for at least routing, and potentially partial processing prior to routing, to the DS management agent.

FIG. 3C is a sequence diagram of an example method for a records integration that includes integrating identified or converted parameter measurements into a record system.

At stage 390A, the metrics values are displayed in the integration module according to a recording system format. At the same time, the integration service, operating on the records system backend, can transfer the metrics values to the records system management service in stage 390B. In turn, the records system management service can forward the metrics values to an insurance policy coordination service, or determine that more information is needed. In the case of the latter, the records management service can store the metrics values until sufficient information has been acquired. In yet another example, the metrics values may be displayed in the records system interface in stage 391 subsequent to stage 390A.

In stage 392, the integration module can issue a request, or otherwise display a request, for authorization to submit metrics value records to the records system, or otherwise provide a selectable option in a GUI. In one example, the GUI for the integration module can be presented within the DS interface as show in FIGS. 5B and 5C. In another example, the integration module can be controlled by the DS integration agent and installed or otherwise operate as a component within the records system interface.

At stage 393, a response to the request, or a selection of an authorization option displayed, in stage 392, can be received. The display of metrics values in stage 390A represents a presentation of metric value-corresponding parameter measurements in a format that is acceptable or stipulated by a records systems. In one example, the format is dictated by reporting requirements established by private and public (government—e.g., Medicare) insurance providers. These reporting requirements often serve as prerequisites for these insurance entities to determine services obtained by subjects are covered by their respective policies and the cost of which should be paid in part or entirely by the insurance entity as a benefit of the subject's policy. In one example, an operator (e.g., a clinician, caregiver, or administrator) with knowledge of specific reporting requirements can view the metrics values displayed in stage 390A and recognize whether the formatted information does or does not include all information required for insurance claim purposes. In practice, this step of review can be secondary to a review process performed by the DS integration agent before providing the measurements and metrics values to the integration module in stage 378. In another example, stages 390A, 392, and 393 may provide an operator with an operator with an opportunity to add non-standard information regarding a subject, the subject's performance of one or more movements or overall performance of a movement session, or any additional comments the operator believes are relevant.

At stage 394, the response received in stage 394 can be transmitted to the integration service and/or the records system management service. As a result of receiving the response, the integration service can process the metrics values and any additional information received through the integration module in stage 395, and generate instructions for integrating the metrics values into a repository of subject history maintained or otherwise updated by the records system. In one example, the integration service can include an indication, value, or tag that is read and used by the records system management service in determining whether or not to submit instructions or otherwise control the insurance policy coordination service to transmit the metrics values in a format required by a particular insurance entity that a subject holds a policy with.

In stage 396, the records system management service implements any aspect of the records instructions requiring action by the records system management service, such as storage in a repository or issuing requests for any additional information, and communicates the records instructions to the insurance policy coordination service. Further, the records system management service forwards any information related to the metrics values that was created or modified since the metrics values were transmitted to the insurance policy coordination service in stage 390B.

As a result of receiving the records instructions and any additional information in stage 396, the insurance policy coordination service process the metrics values in stage 398 according to the records instructions. This could include submitting a report to a relevant insurance entity, a preliminary claim and an indication that additional information will be obtained, or other submission of other informational components specified by protocols specific to a given insurance entity.

FIG. 4 is a sequence diagram of an example method for integrating management of movement sessions with reporting requirements of an electronic records system. More specifically, FIG. 4 illustrates an example method for a situation in which initiation of a device system, and access and use of various device system components such as wearable device groups and/or a DS interface, precede any type of use or accessing of a records system and its components such as a records system interface. Said differently, FIG. 4 represents a sequence in which features of the device system are used for movement sessions as a precursor to uses of device system features to initiate records integration. In contrast, in the exemplary method of FIG. 3A, the record system interface is used to launch the DS interface and provide subject information to the DS backend. In contrast, in the exemplary method of FIG. 4, the DS interface is initiated for a movement session and used to cause execution of a method of records integration corresponding to the exemplary method of FIG. 3C.

At stage 410, wearable device groups, the DS interface, DS management agent, and DS management service implement a train mode for one or more subjects. At stage 414, the DS interface can receive a request to integrate parameter measurements into the record system. As a result, the DS interface can issue a call to the DS integration agent to initiate integration module. The DS integration agent can package the call with authentication information related to an operator and/or a computing device implementing the DS integration agent and from which the call will be transmitted to the integration service in stage 422. At stage 426, the integration service can execute an authentication process to ensure that security of subjects' medical and identity information are not put in jeopardy either from a backend or end user perspective. Thus, the call in stage 422 may include just that information that is required to authenticate that parameter measurements or movement data obtained relates to a subject that the device system is authorized to handle information regarding and observe various state, local, government, or other regulatory agency policies for protecting health information.

Once authenticated in stage 426, the DS integration agent or the integration service can establish a communication channel and launch an integration module in stage 429A or 429B respectively. Where the integration module operates as a component of the DS interface, the DS integration agent can launch the integration. However, where the integration module is a component of record system interface, or includes components in both DS and record system interfaces, the integration service performs the channel establishment with the record system and causes the records system to launch whatever component of the integration module that is part of the record system interface. This backend to backend communication can reflect restrictions that the record system put in place with third party solutions that are not native to the services, agents, databases, or interfaces that make up the record system.

In stage 434, the DS integration agent can identify and convert the parameter measurements in to metric values, with or without communicating with the DS integration service, in like manner to stages 376 and 377 of FIG. 3B. Likewise, the DS integration agent and/or the integration service can transmit the measurements and metrics values to the integration module in stage 434. As a result of receiving the parameter measurements and metrics values, in stage 440, the integration module, integration service, record system management service, and the insurance policy coordination service can implement the records integration of the exemplary method of FIG. 3B.

FIG. 5A illustrates an exemplary graphical user interface (“GUI”) dashboard 500 (“dashboard 500”) for monitoring a movement session of a subject in real time. In one example, a clinician guiding the movement sessions of multiple subjects may be able to view each subject in their own subject-specific dashboard 500. In another example, the operator may be able to select each dashboard for exclusive viewing across an entire area of a display. In another example, the multiple dashboards 500 may be viewed distributed across a display for viewing at the same time. The dashboards may be displayed within a general interface for a device system, or as different instances of a dedicated application, each instance have its own window that can be sized to allow for multiple windows to be shown on a single display.

The dashboard 500 of FIG. 5A includes an exemplary live stream session console 516 (“live console 516”) for use as part of a live stream assisted movement session. The dashboard 500 represents one example of the DS interface that can be provide to and used by a clinician in the methods of FIGS. 2-4. As shown in FIG. 5A, the dashboard 500 can include a navigation menu 502 and a console window 504. The navigation menu 502 includes a plurality of sub-menus, and the console window 504 includes the live console 516.

In the example illustrated in FIG. 5A, mode, record system integration, and messaging sub-menus 506, 508, 510 are shown and designated along with assessment focus, body plane focus, recommendation mode, and movement library sub-menus which are shown but not designated. These sub-menus are disposed above a sub-window 512 that displays a live stream video or a static image of an operator, such as the operator 100 of FIG. 1. In one example, a computing device used by an operator using the dashboard 500 may include a camera that operates during a live streamed movement session, such as the one being depicted in FIGS. 5A and 5B for example.

Each sub-menu may be displayed with the selection of a respective expand option provided next to a title of that sub-menu. When expanded, a given sub-menu can display various primary, secondary, and even tertiary selection options. Selection of certain options, or combinations of options, can cause the dashboard 500 to: change a respective mode; expand other sub-menus; display different sub-menus; expand primary selection options to reveal secondary and tertiary selection options in a hierarchical manner; and/or display different consoles in the console window 504 and/or sub-menus in the navigation menu 502.

In the exemplary case of FIG. 5A, a mode option for a live stream assisted session mode 514 (“live mode 514”) is selected within the mode sub-menu 506. In one example, selection of the live mode 514 can cause the live console 516 to be displayed in the console window 504. The live console 516 can include live stream subject and device feedback windows 518, 520, and expandable movement profile summaries such as exemplary subject movement profiles 522 illustrated in FIG. 5A below the live stream windows 518, 520.

The live stream subject window 518 (“subject window 518”) can include a live stream video of a subject in such detail and clarity, that an operator can observe various aspects of the subject's performance of plan movements. In one example, a first summary section 524 can provide the subject's subject ID or name, and identify the movement being performed.

The live stream feedback window 520 (“feedback window 520”) can include livestream images of wearable devices, such as the first and second wearable devices 112A, 112B being worn by the first subject 120 from FIG. 1 (a live stream video of whom can be con-currently being displayed in the subject window 518 as shown). The feedback window 520 can include a second summary section 526 which includes group ID for the group of wearable devices for which respective delivered feedback is represented in a live stream manner in the feedback window 520. In addition, the second summary section can include a field that shows how many wearable devices are included in the group of devices associated with the ID displayed.

An operator can look at the feedback window 520 and quickly appreciate if all of the wearable devices for that group are operational by comparing the number of devices in the second summary section 526 to the number of devices that are represented in the window. In one example, first and second description boxes 528A, 528B can display messages that describe what feedback each of the first and second wearable devices 112A, 112B is respectively delivering. In another example, where feedback is in the form of flashing lights or text, the same display of lights or text can be recreated within the images of the wearable devices. In other examples, images of the wearable device can also include static text that indicate where a given wearable device is located on a subject, an example of which is shown in FIG. 5A.

In addition, the feedback window 520 can include a selectable feedback edit option 529. In one example, selection the edit option 529 can cause a menu to appear that display how the wearable devices are currently set up to deliver feedback. More specifically, the edit option 529 can be sued to change how wearable devices are configured to deliver customized feedback during non-compliant portions of a physical movement, until a correction by the subject brings the physical movement into compliance with a movement model. A clinician or operator can use the edit option 529 to change the form of feedback (e.g., flashing lights, a flashing screen, sequenced pulses of vibration, sounds, spoken words, etc.) any wearable device in a wearable device group delivers as a subject is performing the movement. In one example, the operator could customize the form of feedback for any device being worn to be based on a proximity of that device's position on a subject, relative to a portion of a limb or limb segment that is not moving in compliance with a movement model.

Each subject movement profile 522 can include a title bar 530 that identifies a portion of a subject's body and movement analysis that summary pertains to and includes. Each profile summary also includes a selectable expand option 532, a results graph 534, a result summary section 538, a selectable comparative analysis option 538. As can be seen in FIG. 5A, the expand options 532 have been selected for first and second subject movement profile 522 for shoulder flexion and should supination respectively. At the same time, the expand option 532 for the third movement profile summary for elbow angle is not selected and this profile is accordingly displayed in a collapsed state.

The graph 534 for each subject movement profile 522 charts a measurable aspect of movement for a body portion of a subject that is the focus of evaluation. Values for one or more evaluation metrics that may correspond to, or be derived from, that measurable aspect, can be determined and included in a result summary section 536 for each movement profile summary 522.

In FIG. 5A, a first evaluation metric 536A for a shoulder is an absolute range of flexion, and a second evaluation metric 536B is a lateral displacement (similar to a swing of a knee) of an elbow relative to a stationary vertical plane arbitrarily established as a point of reference for the purposes of measuring this aspect of a subject' movement. The subject movement profile 522 for shoulder supination includes a supination evaluation metric in a respective result summary section 536. In one example, the values corresponding to these evaluation metrics and displayed in respective results summary sections 536, can represent an average, mean, median value, or another operation. Values for one or more evaluation metrics that may correspond to, or be derived from, a measurable aspect of the subject's movement, can be determined and included in a result summary section 536 for each subject movement profile summary 522. Results for these evaluation metrics can be helpful to an operator or clinician in understanding a subject's ongoing ability to perform a prescribed movement.

As will be understood from the above discussion for stage 382, the subject movement profiles 522, including graphs 534 and result summary sections 536, can be generated real-time as movements are performed by a subject during a movement session. The exemplary display of graphical and numeric data illustrated in FIG. 5A can be continuously updated. That is, as the wearable devices continue to detect movement and transmit movement data, and a DS management agent and/or service processes and characterizes the formatted movement data, the graphs 534 and values for evaluation metrics can be updated and displayed real-time in the live stream console 516 of the console window 504 accordingly.

While guiding a subject's movement session, either in-person or remotely (relative to the subject), an operator or clinician can view the subject in the subject window 518, feedback being delivered in the feedback window 520, and/or how values for evaluations metrics are changing (either with a graphical representation or a discrete value) with the movement profiles 522. An operator standing on the opposite end of a clinic from a subject, or working remotely, may notice from one or all of these portions of the live console 516, something about the subject's movement that needs correction that may be best, expediently, or conveniently achieved through direct instruction from the clinician. The navigation menu 502 provides the operator with the option to send a direct message to the subject with the messaging sub-menu 510.

The messaging sub-menu 510 includes a text field 546 in which messages can be composed, delivery mode options 547, a send option 348A, and a clear option 348B. The delivery mode option can be used by the clinician to direct a message to wearable devices being worn by the subject, or a computing device, such as a phone or laptop, being used by the subject. In this way, the clinician has the option to quickly send a direct message to a subject a time when the clinician is not able to walk across the clinic to speak with the subject because the clinician is guiding another subject. In another scenario, the clinician may be working from a clinic and guiding multiple movement sessions in-person while at same time remotely guiding a movement session of a subject that is at home. The messaging sub-menu 510 can be used to send the subject a message while the clinician helps another subject in the clinic, or in the event that there is an issue with a microphone with a device being used by the clinician or a sound system with a device being used by a subject.

As shown in FIG. 5A, the comparative analysis option 538 is unselected for each of the movement profiles 522. However, just knowing a magnitude of values for evaluation metrics provides may only provide narrow insights into a subject's ability to perform and ability to improve performances of a given movement. More valuable than knowing just a magnitude of values for these evaluation metrics, is knowing how those values compare to values for the same evaluation metrics for a movement profile considered to be good/optimal or bad according to recognized standards of analyzing a type of subject (e.g., human vs. animal, injured vs. athlete, terminal illness vs. no illness).

More specifically, providing a comparative analysis between a subject and another individual who exhibits a bio-mechanically correct movement profile when performing the same movement, may allow a clinician to pinpoint points along a kinematic chain involved in the movement. In turn, the clinician can focus on portions of the subject's body corresponding to these points and quickly make adjustments or provide the subject with instructions for immediate implementation. In recognition of the insights that can come from comparisons discussed immediately above, the dashboard 500 is configured to provide the selectable comparative analysis option 538 for any subject movement profile 522, such as those illustrated in FIG. 5A. An example of the dashboard when at least one comparative analysis option 538 is selected is discussed in detail with reference to FIG. 5B.

FIG. 5B illustrates an exemplary GUI for comparatively monitoring a movement session of a subject in real time using a computing device. As shown, the comparative analysis option 538 for the subject movement profile 522 for shoulder supination is selected. As a result, an exemplary movement comparison summary 550 is also displayed. Each movement comparison summary 550 includes the subject movement profile 522, and a comparison movement profile 552. As the subject profile 522 includes the graph 534 and evaluation metric result summary section 536, the comparison movement profile 552 includes a comparison graph 554 that charts the same measurable aspect as the graph 534. Likewise, the movement comparison profile 552 includes an evaluation metric result summary section 556 that informs an operator of a value of the same evaluation metric reported in the subject movement profile 522.

It will be noted that the comparison movement profile 552 can be generated from a model or simulated performance of a given movement, or a characterization of another individual's performance of the movement. In the case of the latter, the individual movement may be considered optimal by some medical or physical therapy organization, or an individual clinician. Further, data for generating the graph may be obtained in the same way movement data is gathered from a subject. That is, movement data for the individual which provides the basis for a comparative analysis may have been obtained using wearable devices while the individual performed the movement. In one example, positions of the wearable devices on a subject for a movement session or a specific movement, may be the same as the positions of wearable devices on the individual when the individual's movement data was gathered.

Thus, systems and methods of the present disclosure related to the exemplary GUI provide an operator or clinician with a continuously updating visual and empirical representation of a subject's movement next to the same representation of an optimal movement. This can increase a clinician's ability to pinpoint portions of a subject's body and movement for focused modification, treatment, or training. An additional advantage attendant to the comparative analysis feature provided by the dashboard as illustrated in FIGS. 5A and 5B is derived from a clinician or operator's ability to quickly see how a subject's movement profile is specifically changed in response to, or otherwise affected by, a type of feedback being delivered to the subject. As noted above, the subject and comparison movement profiles 522, 552 are updated in real time as a subject performs a movement. Thus, the graphs 534 are continuously updated while the feedback being delivered to the subject is displayed in the feedback window 520. Accordingly, a clinician can quickly see how a subject's movement profile is specifically changed in response to, or otherwise affected by, the type and frequency of feedback that is delivered to the subject.

FIG. 5B also includes a view of an expanded records system integration sub-menu 508 (“integration sub-menu 508”). As shown, integration sub-menu 508 includes selectable link, check, and preview options 558, 560, 562. In one example, selection of the link option 558 can correspond to stage 414 of FIG. 4 in which the DS interface receives a receives a request for records system integration. Further, selection of the link option 558 could cause execution of stages 418, 422, and 429A by a DS integration agent installed on a user device of the clinician. In this example, the link option 558 may be selected before or after a movement session is completed. In the former case, the communication channel may be established with the records system backend to ensure expedient transmission of the metrics values at the conclusion of the movement session.

The check option 560 may be selected or used in situations where a call and launching of the DS interface was expected, but did not occur for some reason. However, selecting the check option 560 can return subject information and initiate the dashboard 500 for a movement session. This may occur, for example in the method of FIG. 3A, where stages 310 to 326 but execution of stage 330 or 334 may be delay due to current workloads/traffic being handled by a records system backend or DS backend, respectively.

The integration sub-menu 508 further includes a text field where notes can be entered, and first option set 566A for performing an action to or with a report formatted according to a format of a records system. As illustrated, an operator or clinician can cause a report to be submitted, stored, or clear according to an option selected from the first option set.

Selection of the preview option 564 from the integration sub-menu 558 can correspond to stage 390A in an example in which an integration module is a component of the DS interface. Results from the selection of this option are discussed in more detail with respect to FIG. 5C.

FIG. 5C illustrates an exemplary UI for generating and reviewing record-system ready data for the purposes of a records integration. FIG. 5C illustrates an exemplary integration console 570 as being presented in the console window 504. In on example, the integration console 570 presents metrics values in a format according to reporting requirements for a records system. The integration console 570 can include metrics tables 572 that include line entries 574 for movements performed during a movement session and metrics categories 580. Prescribed metrics values 582, and achieved metrics values 584 for a current or just-completed movement session (identified as “THIS SESSION”), are include under the categories 580 for the line entries 574. The device system populates the metrics tables 572 the achieved metrics values 584 as identified or converted from parameter measurements. In one example, the prescribed metrics values 582 can be obtained from a movement plan designed through the dashboard. In another example, the prescribed metrics values 582 can be pre-entered by a clinician, or obtained through an information exchanged between the DS integration agent and a third party product used for developing movement plans.

The integration console further includes a second option set 566B for performing an action to or with the formatted report represented in the integration console. Separate from the first and second option sets 566A, 566B, in one example the dashboard can be configured allow a user to edit, remove, or add prescribed and achieved metrics values 582, 584.

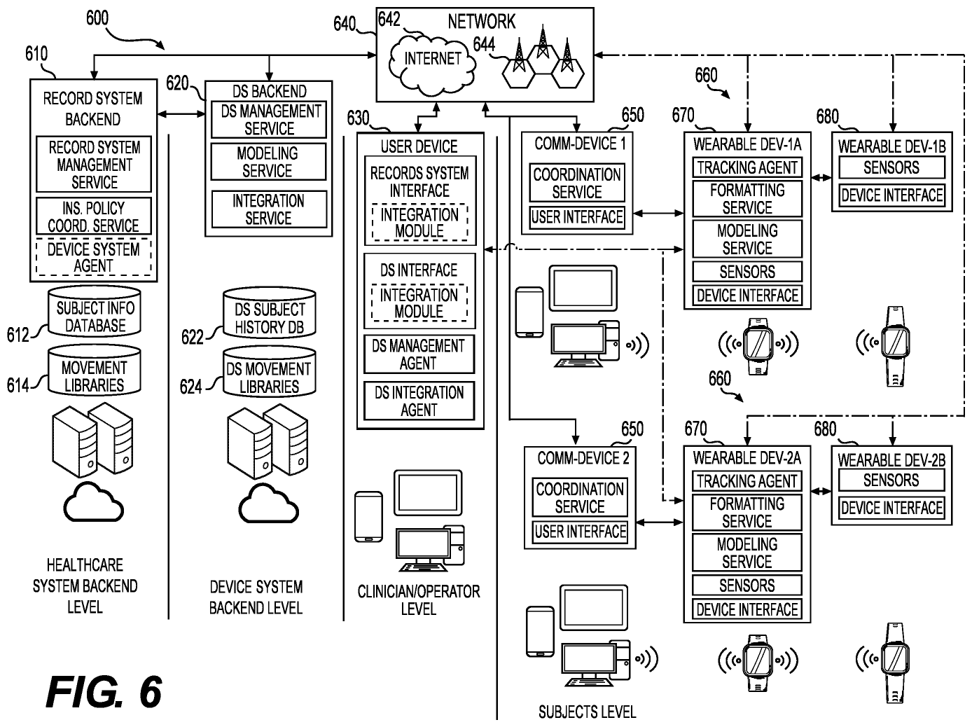

FIG. 6 is an exemplary illustration of system components for integrating movement session management with reporting requirements based on an electronic record system.

The system components 600 can include a record system backend 610, a device system backend 620 (“DS backend 620”), one or more user devices 630, one or more comm-devices 650, and one or more device groups 660 each including at least a first wearable device 660 and a second wearable device 680. System components can be in communication through a diversified network 640 that includes different sub-networks such as the internet 642 and a cellular network 644. In addition, the user device 630, comm-devices 650, first wearable devices 670, and second wearable device 680, can be in direct communications with each other via the wireless communication protocols already mentioned (e.g., Bluetooth, Bluetooth Low Energy, Zigbee, or a Wifi chip).

In one example, the user device 630 and the comm-device 650 are in communication with the diversified network 640 through the internet 642 or a cellular connection 644. The remaining devices can receive information (e.g., movement models, calibrations, messages, movement plans and plan modifications, etc.) through a location/proximity dependent connection with either the user device 630 or the user device 650. For example, in the case of a plan modification being transmitted to a subject from an operator working remotely, the authorization process would occur on with the comm-device 650. Once accepted, the comm-device 650 could reach out to the DS backend 620 or the user device 630 for a new or updated movement model and calibration, and transmit it to a respective first wearable device 660.

In another example, one or both of the wearable devices 670, 680 can include a Wifi chip or cellular connectivity. With the example situation just mentioned, the comm-device 650 could make a call for the model/calibration through the diversified network 640, or instruct the wearable devices 670, 680 to issue a call through the same. Then, the required information and models may be transmitted to the wearable devices 670, 680 through the diversified network without passing through the comm-device 650.

The system components are illustrated in levels to place end user roles into context with how the system components communicate and operate to help end users fulfill those roles. In one example, each of the record system backend 610 and the DS backend 620 can include one or more physical servers, and or cloud-based virtual servers that support the services and agent operating on other system components. In addition, the DS backend 620 can be provided with software-based tools through which an administrator can monitor, manage, update, and modify aspects of, for example, a tracking agent being implemented on one or more wearable devices.

The DS backend 620 is part of a backend system level that supports the operations of services, agents, and applications being implemented on various devices. Such devices, like the user device 630, and in some cases the wearable devices 670, 680 if loaned out to a subject, may be managed devices that the DS backend 620 has direct control over. Other devices, such as the comm-device 650, are likely to be a personal device of and owned by, the subject who uses it. Accordingly, the coordination service of the comm-device 650 may have certain permissions and be configured to generate certain content on the comm-device 650, but ultimate control of the comm-device 650 lies with the subject.

Each of the services running or otherwise being implemented on the DS backend system, clinician/operator, subject, and automation levels, as well as the DS integration agent on the user device can be part of or configured to be compatible with a software product that is at least partially provided by the DS backend 620. The software product can provide tools for system management, communication and coordination, modeling, data formatting, tracking movements, generating components of and supporting selections made through a UI, and any other relevant features.

The record system and DS backends 610, 620 may be supported on a respective backend system level by a respective subject information database 612, 622 and a movement library repository 614, 624. The DS backend 610 may be supported on the backend system level by its own subject information database 622 and movement library repository 614. According to an aspect of the present disclosure, each of these repositories can be a database that is hosted on one or more servers. In another example, one, any, or all of the repositories 612, 614, 622, 624 can be a cloud-based storage component that is part of an infrastructure that includes various computing devices (e.g., user devices and wearable devices) and cloud-based services interconnected by local networks, wide-area networks, wireless communications, and the Internet.

Other examples of the disclosure will be apparent to those skilled in the art from consideration of the specification and practice of the examples disclosed herein. Though some of the described methods have been presented as a series of steps, it should be appreciated that one or more steps can occur simultaneously, in an overlapping fashion, or in a different order. The order of steps presented are only illustrative of the possibilities and those steps can be executed or performed in any suitable fashion. Moreover, the various features of the examples described here are not mutually exclusive. Rather any feature of any example described here can be incorporated into any other suitable example. It is intended that the specification and examples be considered as exemplary only, with a true scope and spirit of the disclosure being indicated by the following claims.

Attached herewith is an Appendix that includes additional descriptions and illustrations of various components, including exemplary: features and examples of wearable devices; a device system; electronic record systems including electronic medical or healthcare record systems; an integration service and interface, a device backend, and an electronic record backend. Any and all examples, features, or other subject matter described or illustrated in the Appendix are encompassed by the present disclosure.

What is claimed is:

1. A method of report generation by integrating movement session management of subjects with reporting requirements based on an electronic health records system (“records system”), the method comprising:
receiving, with a server for a device system, subject information and a request for metrics information from the records system, the subject information corresponding to a subject associated with the metrics information of the request;
generating movement plan options for the subject based on the subject information;
receiving raw sensory data (“RSD”) associated with a performance of movements by the subject, the movements being included in a movement plan selected from the movement plan options, the RSD being obtained by sensors of wearable devices of the device system, the wearable devices being worn by the subject during the performance of the movements;
determining, with a computing device, parameter measurements based on the RSD, the parameter measurements corresponding to performance parameters for movements included in the movement plan;
identifying, by an integration agent executing on one of the server and the computing device, metrics values from the parameter measurements, the metrics values corresponding to reporting metrics required by the record system; and
causing, with at least one of the server and the computing device, a report to be generated according to a format of the records system, the report including at least the parameter measurements identified as the metrics values for the subject.

2. The method of claim 1, further comprising, prior to the receiving the parameter measurements, generating the movement plan based on the subject information and an assessment of the subject using the device system.

3. The method of claim 2, wherein the RSD is generated by the wearable devices implementing a training mode during the movement session.

4. The method of claim 1, further comprising receiving authorization to submit the report with an integration module for the device system, the integration module being implemented as a graphical user interface (“GUI”).

5. The method of claim 4, wherein the integration module transmits and receives information to and from the records system and the device system.

6. The method of claim 5, wherein the integration module is launched within a device system interface.

7. The method of claim 5, wherein the integration module is launched within a records system interface.

8. The method of claim 1, prior to the receiving the parameter measurements, further comprising:
receiving, with an integration service, a request from a records system interface for the reporting metrics;
verifying, with the integration service, information about the subject stored by the records system and the device system; and
requesting, with the integration service, the parameter measurements from the device system based on the verifying.

9. A non-transitory, computer-readable medium comprising instructions that, when executed by a processor, cause the processor to perform stages for report generation by integrating movement session management of subjects with reporting requirements based on an electronic health record (“EHR”) system, the stages comprising:
receiving, with a server for a device system, subject information and a request for metrics information from the records system, the subject information corresponding to a subject associated with the metrics information of the request;
generating movement plan options for the subject based on the subject information;
receiving raw sensory data (“RSD”) associated with a performance of movements by the subject, the movements being included in a movement plan selected from the movement plan options, the RSD being obtained by sensors of wearable devices of the device system, the wearable devices being worn by the subject during the performance of the movements;
determining, with a computing device, parameter measurements based on the RSD, the parameter measurements corresponding to performance parameters for movements included in the movement plan;
identifying, by an integration agent executing on one of the server and the computing device, metrics values from the parameter measurements, the metrics values corresponding to reporting metrics required by the record system; and
causing, with at least one of the server and the computing device, a report to be generated according to a format of the records system, the report including at least the parameter measurements identified as the metrics values for the subject.

10. The non-transitory, computer-readable medium of claim 9, the stages further comprising, prior to the receiving the parameter measurements, generating the movement plan based on the subject information and an assessment of the subject using the device system, wherein the RSD is generated by the wearable devices implementing a training mode during the movement session.

11. The non-transitory, computer-readable medium of claim 9, the stages further comprising receiving authorization to submit the report with an integration module for the device system, the integration module being implemented as a graphical user interface (“GUI”).

12. The non-transitory, computer-readable medium of claim 11, wherein the integration module transmits and receives information to and from the records system and the device system.

13. The non-transitory, computer-readable medium of claim 12, wherein the integration module is launched within one of a device system interface and a records system interface.

14. The non-transitory, computer-readable medium of claim 9, prior to the receiving the parameter measurements, the stages further comprising:
receiving, with an integration service, a request from a records system interface for the reporting metrics;
verifying, with the integration service, information about the subject stored by the records system and the device system; and
requesting, with the integration service, the parameter measurements from the device system based on the verifying.

15. A system for report generation by integrating movement session management of subjects with reporting requirements based on an electronic health records system (“records system”), the system comprising:
a device system including a server and wearable devices; and
a computing device;
wherein the device system and the computing system include:
at least one a memory storage including a non-transitory, computer-readable medium comprising instructions, and
at least one processor that executes the instructions; and
wherein the at least one processor executes the instructions to perform stages comprising:
receiving, with the server for the device system, subject information and a request for metrics information from the records system, the subject information corresponding to a subject associated with the metrics information of the request,
generating movement plan options for the subject based on the subject information,
receiving raw sensory data (“RSD”) associated with a performance of movements by the subject, the movements being included in a movement plan selected from the movement plan options, the RSD being obtained by sensors of the wearable devices of the device system, the wearable devices being worn by the subject during the performance of the movements,
determining, with the computing device, parameter measurements based on the RSD, the parameter measurements corresponding to performance parameters for movements included in the movement plan,
identifying, by an integration agent executing on one of the server and the computing device, metrics values from the parameter measurements, the metrics values corresponding to reporting metrics required by the record system, and
causing, with at least one of the server and the computing device, a report to be generated according to a format of the records system, the report including at least the parameter measurements identified as the metrics values for the subject.

16. The system of claim 15, the stages further comprising, prior to the receiving the parameter measurements, generating the movement plan based on the subject information and an assessment of the subject using the device system, wherein the RSD is generated by the wearable devices implementing a training mode during the movement session.

17. The system of claim 15, the stages further comprising receiving authorization to submit the report with an integration module for the device system, the integration module being implemented as a graphical user interface (“GUI”).

18. The system of claim 17, wherein the integration module transmits and receives information to and from the records system and the device system.

19. The system of claim 18, wherein the integration module is launched within one of a device system interface and a records system interface.

20. The system of claim 15, prior to the receiving the parameter measurements, the stages further comprising:
receiving, with an integration service, a request from a records system interface for the reporting metrics;
verifying, with the integration service, information about the subject stored by the records system and the device system; and
requesting, with the integration service, the parameter measurements from the device system based on the verifying.
